(19) United States
(12) Patent Application Publication
(10) Pub. No.: US 2024/0000446 A1
(43) Pub. Date: Jan. 4, 2024

(54) DEVICE FOR THE TREATMENT OF STRESS URINARY INCONTINENCE

(71) Applicant: Oxford University Innovation Limited, Oxford (GB)

(72) Inventor: Luke LINTIN, Oxford (GB)

(21) Appl. No.: 18/039,496

(22) PCT Filed: Dec. 1, 2021
(86) PCT No.: PCT/GB2021/053133

§ 371 Date: May 31, 2023

(30) Foreign Application Priority Data
Dec. 3, 2020 (GB) 2019103.7

(51) Int. Cl. A61B 17/04; A61B 17/06; A61B 90/00

(52) U.S. Cl. CPC A61B 17/0401; A61B 17/06004; A61B 90/37; A61B 2017/00805

(57) ABSTRACT

A needle (16) for deploying a suture is provided. The needle (16) has a feeding end (11) with an aperture (19) for receiving the suture and a pointed end (3) for creating an incision. The needle (16) extends between the feeding end (11) and the pointed end (3). The needle (16) also has a second aperture (18) and an interior channel (5) extending between the first aperture (19) and the second aperture (18) for transporting the suture from the first aperture (19) to the second aperture (18). A kit for deploying sutures includes at least one suture, at least one needle (16) and at least one pusher (12). The pusher (12) pushes the suture through the interior channel (5) of the needle (16) and out of the second aperture (18).

The present invention relates to a device for the treatment of stress urinary incontinence in women, and also to a kit and a method of treatment using the needle.

Stress urinary incontinence (SUI) most commonly arises a result of the urethra not being sufficiently supported by the pelvic floor muscles and fascia. SUI is known to affect 13% to 48% of women. Surgical intervention can become necessary when conservative measures that are recommended for the treatment of stress urinary incontinence do not work. The options currently available to patients include: colposuspension, autologous slings, synthetic mid-urethral slings, artificial sphincters and the implantation of urethral bulking agents.

In recent years, one of the common treatment options for SUI— synthetic mid-urethral sling procedures (vaginal mesh surgery)—has become controversial, as reports of female patients being injured by the procedure became public. Vaginal mesh surgery involves inserting a strip of synthetic mesh, behind the urethra, for support. Problems such as permanent nerve damage, pain, incontinence and constipation were reported by patients who had received the treatment.

In 2018, the UK's National Health Service temporarily halted mesh operations to investigate these apparent problems. As a result of the pause on vaginal mesh surgery, the formerly common procedure became a rare, last-resort option. This has significantly reduced the number of surgical options available to patients for treating SUI.

An alternative to vaginal mesh surgery is colposuspension. In colposuspension surgery, the periurethral region is lifted and held in place with stitches. Colposuspension may involve open surgery, necessitating a large abdominal incision. It is commonly performed under general anaesthetic and recovery time typically takes a minimum of six weeks. A more limited number of surgeons have the skill and expertise to perform the more technical laparoscopic (keyhole) colposuspension surgery. Even laparoscopic colposuspension requires general anaesthetic and, typically, patients remain in the hospital for a number of days following the procedure. Similarly to the open surgery option, recovery from laparoscopic colposuspension takes up to six weeks.

The only options currently available for patients seeking short recovery times involve injecting synthetic material into the body—e.g. urethral bulking injections. However, urethral bulking injections do not yield the same level of long term success compared to the other options—e.g. colposuspension and autologous slings which are more invasive.

It is an aim of the present invention to provide an improved apparatus and procedure for the treatment of stress urinary incontinence.

According to a first aspect the present invention provides a needle for deploying a suture, the needle comprising:
a feeding end comprising a first aperture for receiving the suture;
a pointed end for creating an incision, wherein the needle extends between the feeding end and the pointed end;
a second aperture proximal to and spaced from the pointed end;
an interior channel extending between the first aperture and the second aperture for transporting the suture from the first aperture to the second aperture.

The invention provides a needle which can insert a suture into a suitable or desired location—e.g. in a human or animal body. The needle has two ends: a feeding end and a pointed end, and has an interior channel through the needle, from a first aperture in the feeding end to a second aperture proximal to, but spaced from, the pointed end. This allows the suture to be introduced (fed) into the interior channel at the feeding end and to be transported (e.g. pushed) through the interior channel from the first aperture, toward the second aperture from which the suture can exit. The particular distance between the pointed end and the second aperture allows the suture to exit at a desired position spaced from the pointed end.

Thus it will be appreciated that the needle is configured to deliver sutures at a particular distance from the pointed end. This helps a practitioner to deliver a suture, e.g. a T-bar suture, to a desired location in a target tissue (e.g. within the body), e.g. from knowledge and placement of the location of the needle's pointed end. This may allow less invasive percutaneous procedures to be performed, using the needle, for the placement of sutures within tissue.

In a set of embodiments, the needle comprises a needle body longitudinally extending between the feeding end to the pointed end. Preferably, the needle body is substantially cylindrical, e.g. at least between the first and second apertures.

In a set of embodiments, the needle body is rigid. Preferably the needle body is straight, i.e. the needle (body) extends in a straight line between the feeding end and the pointed end. In a set of embodiments, the needle body is curved.

The transverse cross-section of the needle body (e.g. in a plane perpendicular to the direction in which the needle extends longitudinally between the feeding end and the pointed end) may have any suitable or desired shape. In a preferred set of embodiments, the needle body has a circular transverse cross-section. In another set of embodiments, the needle body has an elliptical (i.e. having non-zero eccentricity) transverse cross-section.

Similarly, the transverse cross-section of the interior channel (e.g. in a plane perpendicular to the direction in which the needle extends longitudinally between the feeding end and the pointed end) may have any suitable or desired shape. In a set of embodiments the interior channel has a circular transverse cross-section. In another set of embodiments, the interior channel has an elliptical (i.e. having non-zero eccentricity) transverse cross-section.

A portion of the interior channel may extend longitudinally through the needle body along the same longitudinal axis as the needle body. Thus, in a set of embodiments, at least a portion of the interior channel and the external surface of the needle body are coaxial.

Preferably the thickness of the wall of (at least a portion of) the needle body (the wall being defined by the inner and outer surfaces of the needle body) is substantially uniform (e.g. around the perimeter and/or along the length of the needle body, in a radial direction). The wall of the needle body has any suitable and desired thickness. In a set of embodiments, the wall has a thickness (in the radial direction between the inner and outer surfaces of the needle body) between 0.05 mm and 1 mm, e.g. between 0.3 mm and 0.5 mm.

In a set of embodiments, the needle body has a length (in the longitudinal direction of the needle between the feeding end and the pointed end) which is greater than its width (in a direction perpendicular to the longitudinal direction). Preferably the length of the needle body is between 20 and 150 times greater than the width of the needle body, e.g. between 50 and 100 times.

Preferably the needle body has a width between 1.5 mm and 5 mm. Preferably the needle body has a length between 100 mm and 300 mm, e.g. between 150 mm and 250 mm.

In a set of embodiments, the pointed end comprises a bevel. In other words, the pointed end of the needle body terminates at a slant (relative to the longitudinal axis of the needle body). For example, if the needle body has a circular transverse cross-section, then a slanted end forms an elliptical face at the pointed end.

The angle of the distal tip of the pointed end (e.g. formed between the slanted (e.g. elliptical) face and the perimeter of the needle body) may be between 12 and 45 degrees, e.g. between 25 and 30 degrees.

The second aperture may be located at any suitable and desired position relative to the pointed end. In a set of embodiments, the second aperture extends through the wall of the needle body, e.g. at a particular (defined) distance from the pointed end. Thus preferably the second aperture is located on a side of the needle body. When the pointed end comprises a bevel, preferably the second aperture is spaced from the bevel.

The second aperture may be spaced from the pointed end by any suitable and desired distance. The particular distance between the pointed end and the second aperture may be between 1 mm and 20 mm, e.g. between 2 mm and 10 mm, e.g. between 3 mm and 6 mm.

The particular distance between the pointed end and the second aperture may, for example, be measured from the distal tip of the pointed end to the edge of the second aperture most proximal to the pointed end.

In a set of embodiments, the needle comprises markings (e.g. proximal to the feeding end) indicating the side of the needle in which the second aperture is formed. This helps the user to know in which direction the suture will exit the second aperture when the second aperture is not visible—e.g. when the needle is inside the human or animal tissue.

The particular distance between the second aperture and the pointed end may be set based on a number of factors. In a preferred embodiment, the particular distance is based on the thickness of a target human or animal tissue and/or the angle at which the end of the suture exits from the second aperture into that target tissue.

In a set of preferred embodiments, the portion of the needle between the pointed end and the second aperture is closed—i.e. the interior channel does not extend through this portion. This portion may be hollow. However, in a set of embodiments, the portion of the needle between the pointed end and the second aperture is solid.

In a set of embodiments, the interior channel comprises a deflection surface arranged to deflect a suture toward the second aperture (from the interior channel) when the suture is incident on the deflection surface. In such a set of embodiments, the point where the second aperture meets the deflection surface is on the side of the second aperture proximal to the pointed end of the needle (e.g. opposite the side of the second aperture proximal to the feeding end of the needle). The deflection surface is preferably arranged to guide the suture out of interior channel through the second aperture. The deflection surface may comprise one side of the wall of the interior channel (i.e. the inner surface of the needle body), which preferably forms a curve—e.g. a smooth curve—toward the edge of the second aperture most proximal to the pointed end of the needle. Thus preferably the deflection surface extends between the wall of the interior channel opposite the second aperture to the edge of the second aperture proximal to the pointed end of the needle.

In a set of embodiments, the side of the wall of the interior channel opposite to the deflection surface is parallel to the longitudinal axis of the needle body—i.e. having no curvature (in the longitudinal direction)—between the feeding end and the edge of the second aperture most proximal to the feeding end.

Each of the first and second apertures may have any suitable and desired size and shape; however, in a preferred set of embodiments the second aperture is circular or elliptical (i.e. having a non-zero eccentricity). The first aperture may be elliptical or circular.

The needle (body) may comprise a receiving portion at the feeding end of the needle arranged to receive the suture into the feeding end of the needle. The receiving portion may surround the needle body at the feeding end. In a set of embodiments, the receiving portion comprises or surrounds the first aperture. The receiving portion may be any particular or desired shape. In a set of embodiments, the receiving portion is tapered (e.g. substantially conical) having a narrower end and a wider end. In a set of embodiments, the narrower end is oriented in the direction of the pointed end of the needle, with a wider base at the feeding end. The wide base may further comprise a lip—e.g. the wide base extends out into a larger disc shape. In a preferred set of embodiments, the conical receiving portion has its longitudinal axis aligned with the longitudinal axis of the interior channel.

When the receiving portion has its wider end at the feeding end of the needle, e.g. such that the wider end defines or surrounds the first aperture, the first aperture may itself be tapered within the receiving portion, with a wider end of the first aperture at the feeding end and a narrower end extending into the interior channel. This may help the user to introduce the suture and/or the pusher into the relatively narrow bore of the interior channel. However, in some embodiments the width (e.g. diameter) of the first aperture is substantially equal to the width (e.g. diameter) of the interior channel. Here, the receiving portion may be provided for more ergonomical reasons, to assist the user when handling the needle.

In a set of embodiments, the wider end (base) of the receiving portion has a diameter at least three times, e.g. at least six times, greater than the narrower end. In a set of embodiments, the narrower end of the receiving portion has the same width (e.g. diameter) as the needle body, e.g. it tapers from the wider end down to the needle body.

In some embodiments, laser crosshairs may be projected onto a human or animal body to signpost the needle entry point. In such embodiments, the wider end of the receiving portion helps to provide a projection surface such that the laser crosshairs may be more easily aligned with the first aperture.

When the needle comprises markings indicating the side of the needle in which the second aperture is formed, the receiving portion may comprise the markings.

The needle may be made from stainless steel or any other suitable metal or (metal) alloy. In a set of embodiments, the needle is composed of a material that is visible through fluoroscopic imaging and/or computed tomography (CT) imaging.

In a set of embodiments, the needle is arranged to deploy T-bar sutures. T-bar sutures may be easily fixedly held into place when deposited into soft tissue and, if bio-absorbable, may stay secure by the formation of scar tissue.

The needle may be arranged to deploy non-absorbable sutures. In a set of embodiments, the needle is arranged to deploy partially and/or completely absorbable sutures.

In a set of embodiments, the needle is arranged to receive a pusher arranged to push the suture through the interior channel of the needle and out of the aperture.

The needle, the suture and the pusher may be supplied separately. In one embodiment, however, the needle, the suture and the pusher are supplied together as a kit. Thus when viewed from a second aspect the invention provides a kit for deploying sutures, comprising:
at least one suture;
at least one needle according to the first aspect of the invention; and
at least one pusher, arranged to push the suture through the interior channel of the needle and out of the second aperture.

Kits according to the second aspect of the invention may include one or more (e.g. all) of the features described in accordance with the other aspects and embodiments of the invention.

In a set of embodiments, the sutures are T-bar sutures. In a set of embodiments, each suture comprises a footplate portion and a thread portion. The footplate portion is preferably rigid and rod-shaped. In a set of embodiments, the thread portion extends from approximately halfway across the longitudinal length of the footplate portion (e.g. away from the footplate portion in a substantially perpendicular direction). T-bar sutures may be easily fixedly held into place when deposited at the desired location, e.g. into soft tissue, through anchoring of the footplate.

The suture may be non-absorbable; however, in a set of embodiments, the suture is partially made from absorbable materials. In a set of embodiments, when the suture is a T-bar suture, the footplate portion of the T-bar suture is absorbable. For example, when the footplate portion of the suture is absorbable, then the thread portion of the suture may stay sufficiently anchored into the target tissue owing to the formation of scar tissue. The newly formed scar tissue may thus hold the thread in place in lieu of the footplate portion—i.e. after the footplate has been absorbed by the body. In another set of embodiments, the (whole) suture is made from entirely absorbable materials. The footplate of the T-bar suture may also be made from non-absorbable material (e.g. stainless steel).

In a set of embodiments, the (e.g. footplate portion of the) suture comprises an absorbable material comprised of approximately a 50:50 blend of lactide and glycolide polymers.

In a set of embodiments, the kit comprises a member for inserting into an orifice (e.g. the vagina) of the human or animal body, arranged to support the pointed end of the needle in the target tissue. In a set of embodiments, the member is substantially rigid. Preferably, the member has a resilient outer surface, e.g. that is not easily perforated by the needle. For example, the member may be solid. The member may be hollow, e.g. being made of a resilient material that is arranged to resist perforation by the needle. In a set of embodiments, the member is made from a biocompatible material. In a set of embodiments the member is made from a polymer, e.g. high density plastic or another material with partially radiopaque properties.

The member may be any suitable or desired shape or size. In one set of embodiments, the member is elongate (e.g. its length is greater than its width). In one set of embodiments, the member is cylindrical, e.g. with a (e.g. smoothly) tapered end. The tapered end may provide a degree of comfort when inserted into the orifice. In a set of embodiments, the member comprises an ergonomic handle e.g. a grip. The member may have a length between 200 mm and 400 mm, e.g. between 250 mm and 350 mm.

In a set of embodiments, the suture comprises a free end. The free end being the end of the suture not held fixedly in place in the target tissue—e.g. the end of the thread portion of a T-bar suture distal to the footplate portion. In a set of embodiments, the kit comprises an anchoring device for securing the free end of each suture under tension at an anchoring point.

The anchoring device may be made from any suitable material. In a set of embodiments, the anchoring device is made from a bio-absorbable material—e.g. collagen. In another set of embodiments, the anchoring device is made of stainless steel. In a set of embodiments, the anchoring device is a suture locking device or crimp.

In a set of embodiments, the kit comprises a tunnelling device (e.g. a subcutaneous tunnelling device), for pulling the thread portion or free end of each suture through tissue to an anchoring point. In a set of embodiments, more than one suture (thread portion or free end) is tunneled to a common anchoring point. In a set of exemplary embodiments, where a first suture is deployed at the left side of the vaginal wall and a second suture is deployed at the right side of the vaginal wall, suture ends from each side (left and right) are tunneled to a midline incision where they may be tied together.

In a set of embodiments, all materials left behind following the procedure are absorbable—e.g. the suture footplate, the suture thread and the anchoring device.

The pusher may be any suitable size and shape. In a set of embodiments, however, the pusher comprises a rod-shaped portion sized and shaped to fit within the interior channel of the needle. In a set of embodiments, the length of the rod-sized portion of the pusher is such that the suture can be pushed all the way out of the second aperture by the pusher, but not too long such that the pusher protrudes out of the second aperture.

For example, the rod-shaped portion of the pusher may have a length substantially equal to the length of the needle subtracted by the particular distance between the pointed end and the second aperture, i.e. substantially equal to the length of the interior channel of the needle. In one set of embodiments, the rod shaped portion of the pusher may have a length between 80 mm and 500 mm, e.g. between 150 mm and 250 mm.

The rod-shaped portion of the pusher may be made from stainless steel, Angiography wire, or any other suitable alloy.

The pusher may comprise a disc shaped portion at one end. This may help the practitioner handle the pusher, such that the pressure on the user's hand is reduced by exerting a pushing force across a greater surface area.

The kit may further comprise one or more (e.g. all) of the following: a gaily pot and/or sponges for sterilising the skin; surgical drapes having apertures in the groin region and/or vaginal region; a scalpel—e.g. a no. 11 blade; a pair of sterile scissors; artery clips for holding sutures; a sharps box; a syringe and/or long hypodermic needles for local anaesthetic infiltration; a urinary catheter and/or syringe with water for a catheter balloon; sterilising swabs and/or sterile drapes.

In the case where a catheter is used, the kit may further comprise a metal wire, e.g. for placing inside the urethral catheter to enhance visibility of the urethra under medical imaging—e.g. fluoroscopy or computed tomography (CT). Equally, the kit may comprise a liquid contrast agent, e.g. for feeding into the catheter to enhance visibility of the urethra.

For the treatment of stress urinary incontinence the needle and/or kit described herein may be used, in particular, to perform a procedure to provide support to the pubourethral ligament. This is considered novel and inventive in its own right.

Thus when viewed from a third aspect, the invention also provides a method for the treatment of stress urinary incontinence, the method comprising:
inserting a needle according to the first aspect of the invention into a female human or animal body;
advancing the needle until the pointed end reaches the periurethral anterior vaginal wall tissue;
transporting the suture through the interior channel of the needle until the end of the suture exits the needle through the second aperture and the suture is retained in the periurethral anterior vaginal wall tissue;
removing the needle from the female human or animal body; and
securing the suture, under tension such that the pubourethral ligament is reinforced.

When used for the treatment of stress urinary incontinence, the needle may be inserted at the groin, with the pointed end directed towards the target tissue, the periurethral tissue in the vaginal wall. Once in position, with the pointed end at the vaginal wall, the suture is transported through the needle to be deployed into the soft tissue of the vaginal wall, with the particular distance between the pointed end and the aperture helping to ensure that the suture exits the needle at a particular distance from the inner surface of the vagina such that the vaginal wall is not punctured by the suture or the needle. In other words, the spacing between the pointed end and the aperture helps to ensure that the suture does not pass through the vaginal wall into the vaginal lumen. However, in some embodiments the suture (e.g. footplate of a T-bar suture) may be placed within the vaginal lumen for additional support.

After the suture is anchored at the one end in the target tissue, the other (free) end of the suture can then be secured under tension, reinforcing the pubourethral ligament, thus providing a similar effect to colposuspension. The suture may be secured superficially—i.e. near to the skin—or tunnelled anterior to the pubic symphysis.

The procedure of the third aspect may thus be able to provide urethral support, in a much less invasive way than colposuspension, for example, with accurate suture placement. The delivery of the suture through the needle may therefore enable treatment of SUI through a day-case procedure requiring only local anaesthetic. The procedure may thus be a cheaper, faster and less morbid procedure.

It will be appreciated that the invention helps to provide more minimally invasive procedures to treat stress urinary incontinence that require much shorter recovery times. Furthermore, largely due to the reported mal-effects of synthetic mid-urethral slings, a day-case procedure which can treat stress urinary incontinence while leaving little to no synthetic material behind in the vagina has a number of benefits.

Methods according to the third aspect of the invention may include one or more (e.g. all) of the features described in accordance with the other aspects and embodiments of the invention.

In a set of embodiments, the suture is a T-bar suture. T-bar sutures are typically designed to be placed in a lumen, e.g. stomach. In embodiments of the present invention, the needle and kit are designed to allow T-bar sutures to be placed within the soft tissues. As long as the T-bar suture footplate anchors within the tissue, and the other end of the suture can be anchored/secured under tension, then the procedure may have a similar (e.g. substantially the same) effect as colposuspension—i.e. reinforcement of the pubourethral ligament.

The needle could be determined to be in the correct position in any suitable or desired way—e.g. using imaging. Preferably, the method comprises inserting a member into the vagina of a female human body. The member may, for example, be inserted into the vagina before the needle is introduced into the female human body. The needle could be determined to be in the correct position, for example, when the pointed end is in contact with the member—e.g. the practitioner may feel some resistance to the advancement of the needle owing to this contact.

In a set of embodiments, the method comprises maintaining pressure between the pointed end of the needle and the member while the suture is transported through the needle and (at least) until the end of the suture exits the needle. This may help to maintain the accuracy of the placement of the second aperture by reducing movement of the needle in relation to the member (and thus the tissue into which the suture is being deployed).

The suture may be transported through the interior channel of the needle in any suitable or desired way. In a set of preferred embodiments, the method comprises using a pusher to push the suture through the interior channel needle. The method may comprise feeding the pusher into the needle and through the interior channel until the end of the suture exits the needle through the second aperture. The method may comprise removing the pusher from the needle and/or the female human body, e.g. after the suture is inserted and retained in the periurethral anterior vaginal wall tissue.

In a set of embodiments the method comprises imaging the needle using fluoroscopic imaging and/or computed tomography (CT) imaging. For example, fluoroscopic imaging may help guide the needle to the target location.

In a set of embodiments, the method comprises using a catheter (e.g. inserting a catheter into the urethra). In such a set of embodiments, the method may further comprise inserting a metal wire inside the urethral catheter, e.g. to enhance visibility of the urethra under medical imaging—e.g. fluoroscopy or computed tomography (CT). In set of embodiments, the method may comprise feeding a liquid contrast agent into the catheter to enhance the visibility of the urethra—e.g. when using medical imaging.

In a set of embodiments, the method comprises rotating the needle, about its longitudinal axis, and deploying another suture through the needle into the tissue. The needle may be rotated, approximately 180° about its longitudinal axis before another suture is deployed.

The suture may be secured under tension in any suitable or desired way. In a preferred embodiment, the method comprises securing the thread portion of the suture with an anchoring device. For example, the thread portion of the suture may be tensioned by hand, whilst the anchoring device (e.g. a crimp) is pushed over the suture, e.g. as deep as it will go into the tissues until resistance is met. The anchoring device may then be crimped (to maintain the tension in the suture). In a set of embodiments, at least one anchoring device is placed into the soft tissues of the groin and/or under the skin (e.g. when two sutures are tunneled to the midline and crimped together).

For the treatment of stress urinary incontinence T-bar sutures may be used, in particular, to provide support to the pubourethral ligament. This is considered novel and inventive in its own right.

Thus when viewed from a fourth aspect, the invention also provides a method for the treatment of stress urinary incontinence, the method comprising:
transporting a T-bar suture into the periurethral anterior vaginal wall tissue of a female human or animal body such that the T-bar suture is retained in the periurethral anterior vaginal wall tissue; and
securing the T-bar suture, under tension such that the pubourethral ligament is reinforced.

Methods according to the fourth aspect of the invention may include one or more (e.g. all) of the features described in accordance with the other aspects and embodiments of the invention. Preferably the T-bar suture comprises a footplate portion and a thread portion, wherein the footplate portion of the T-bar suture is retained in the periurethral anterior vaginal wall tissue and the T-bar portion of the suture is secured.

Features of any aspect or embodiment described herein may, wherever appropriate, be applied to any other aspect or embodiment described herein. Where reference is made to different embodiments, it should be understood that these are not necessarily distinct but may overlap.

One or more non-limiting examples will now be described, by way of example only, and with reference to the accompanying figures in which:

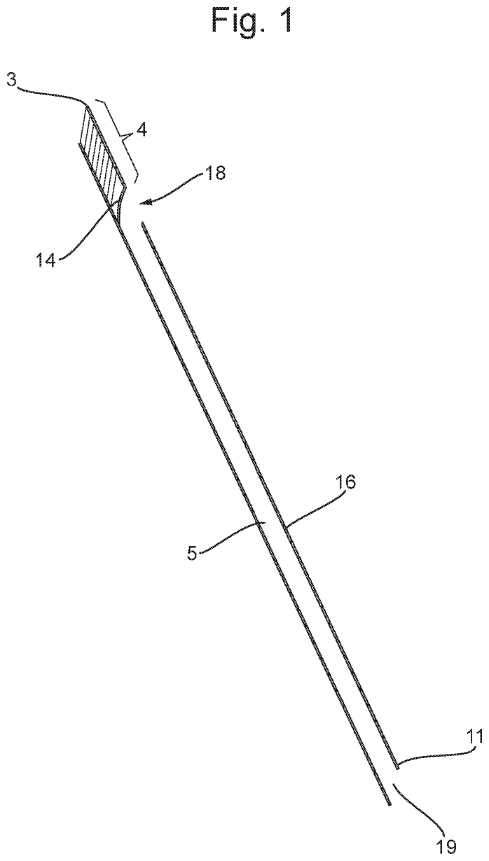

FIG. 1 schematically shows a longitudinal cross-section of a needle embodying the invention;

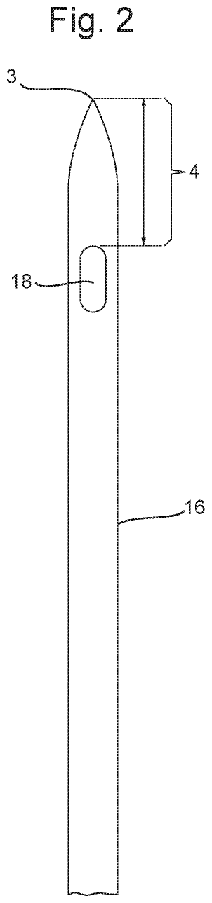

FIG. 2 schematically shows the needle of FIG. 1 in the plane of the aperture;

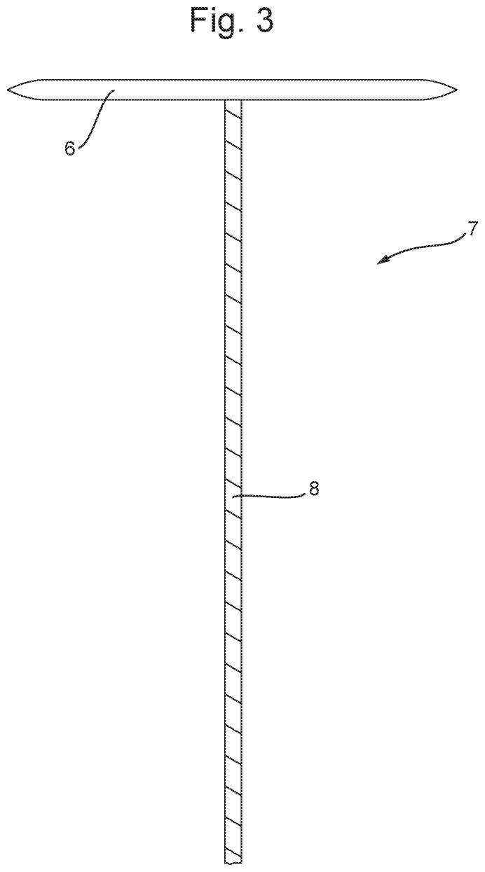

FIG. 3 shows a T-bar suture for use with the needle shown in FIGS. 1 and 2;

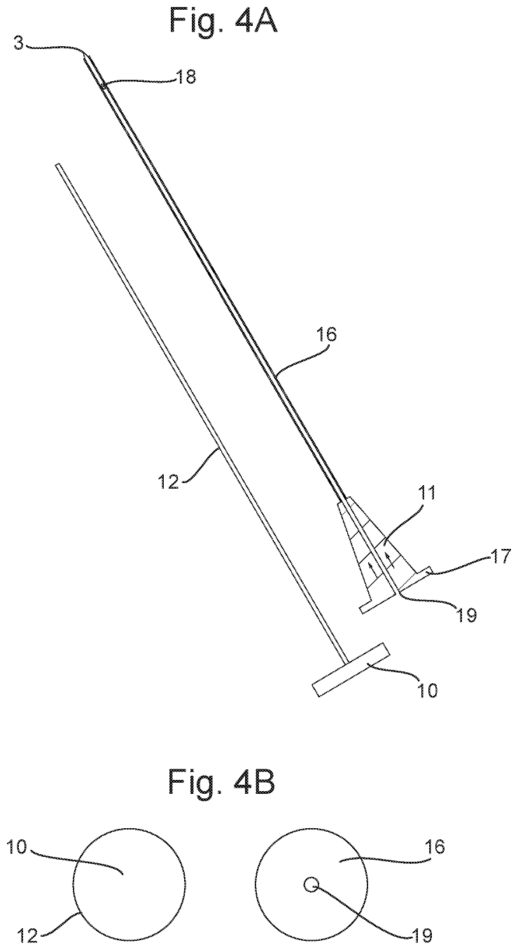

FIGS. 4A and 4B show in more detail a variant of the feeding end of the needle and a pusher;

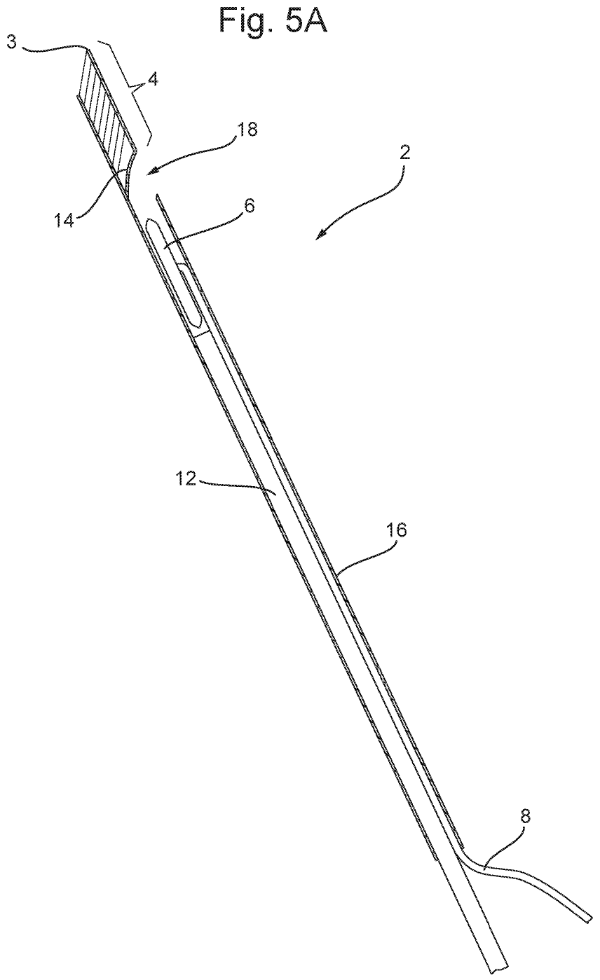

FIGS. 5A and 5B show schematically a longitudinal cross-section of the delivery of the T-bar suture of FIG. 3 through and out of the needle of FIG. 1;

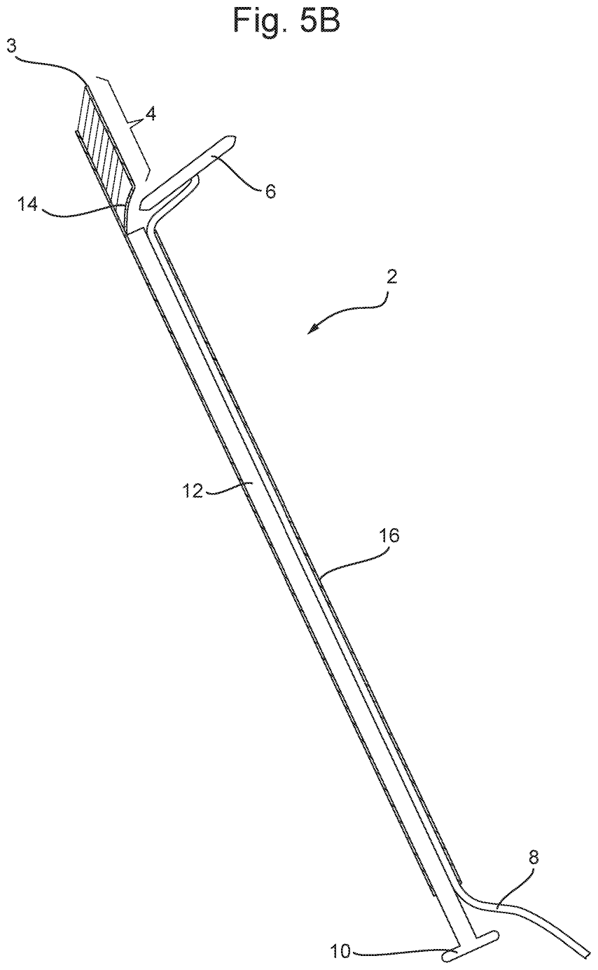

FIG. 6 shows schematically a procedure using the needle of FIG. 1;

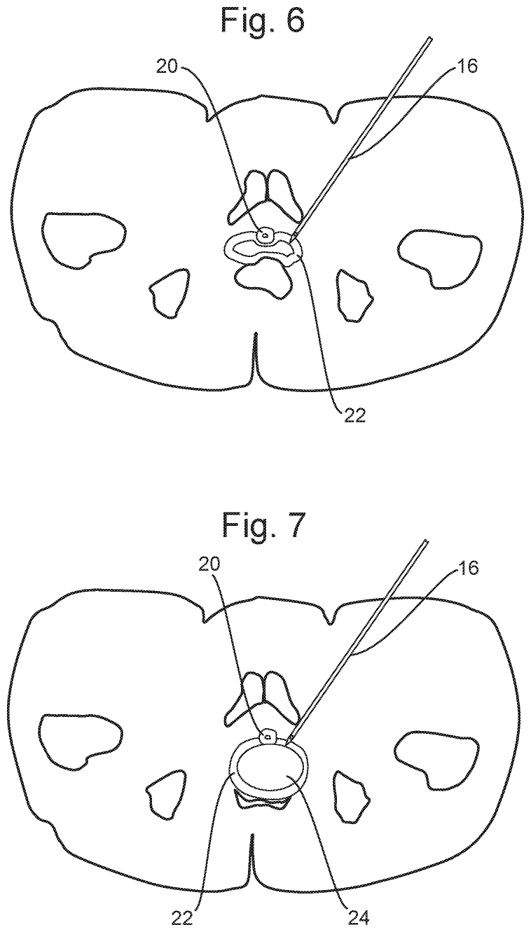

FIG. 7 shows schematically a variant of the procedure shown in FIG. 6;

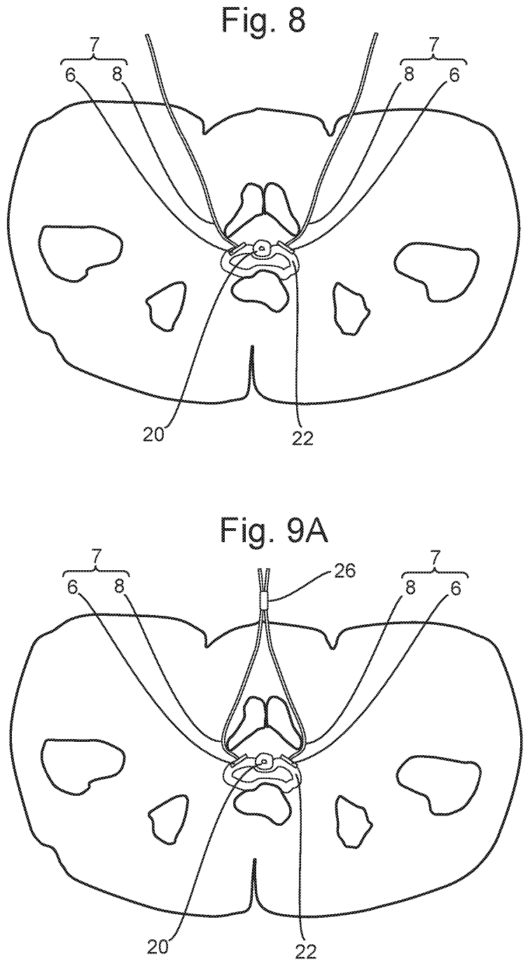

FIG. 8 shows schematically a later stage of the procedure compared to FIGS. 6 and 7;

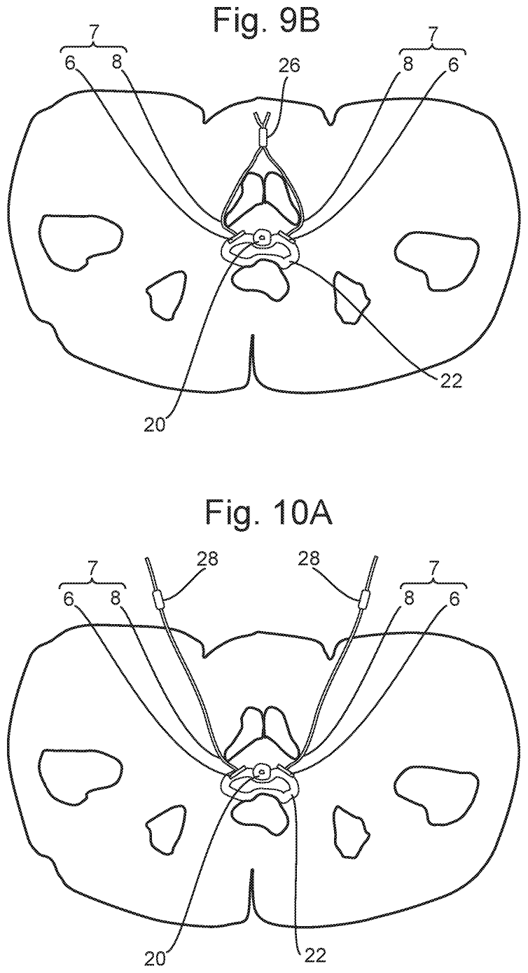

FIG. 9A shows schematically a first method of securing the free end of each suture together with a common crimp;

FIG. 9B shows schematically the final position of the secured sutures according to the method of FIG. 9A;

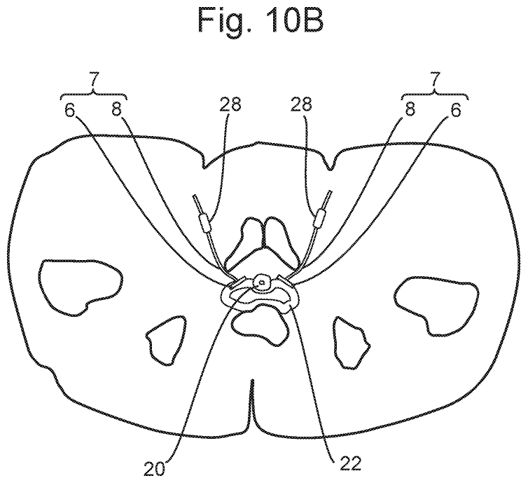

FIG. 10A shows schematically a second method of securing the free end of each suture separately with a dedicated crimp for each suture; and

FIG. 10B shows schematically the final position of the secured sutures according to the method of FIG. 10A.

FIG. 1 shows a longitudinal cross-section of a needle according to an embodiment of the present invention. The stainless steel needle 16 has an interior channel 5 that extends between a first aperture 19 at a feeding/handling end 11, through the needle to a second aperture (side-hole) 18. The side-hole 18 is spaced from the pointed end 3 of the needle. The distance 4 between the pointed end 3 of the needle 16 and the side-hole is set to suit the intended procedure, such that the final position of the side-hole 18 reaches a certain depth in the target human or animal tissue, or a certain distance from a particular part of the body, when the needle 16 is inserted into a human or animal body. The embodiment in FIG. 1 shows one side wall of the an interior channel 5 of the needle 16 (the side furthest from the side-hole 18) having a surface 14 that curves toward the side-hole 18.

In FIG. 2, an alternate view of the needle is shown. The schematic shows the needle in a plane facing the side-hole 18. The pointed end 3 of the needle 16 and uppermost point of the aperture 18 are separated by a particular distance 4. The side-hole 18 is sized and shaped such that a suitable suture can be delivered from the needle 16, exiting through the side-hole 18.

Although the needle 16 is suitable for deploying any type of suitable suture, one preferred type of suture 7 is shown in FIG. 3. The T-bar suture 7, shown in FIG. 3, comprises a rigid bar 6, herein referred to as the ‘footplate’. From the centre of the footplate 6, i.e. from the middle of the longitudinal axis of the bar 6, extends a thread 8. The T-bar suture 7 is designed to anchor into soft tissue such that when tension is applied to the thread 8, the footplate does not slip or move.

In FIG. 4A a full view of the needle 16 and a pusher 12 is shown. The pusher 12 is sized and shaped so that it fits within the interior channel 5 of the needle 16. The length is such that when fully inserted into the needle 16, the end of the pusher 12 reaches the aperture 18 situated towards the pointed end 3 of the needle 16. In this embodiment, the pusher 12 is a stiff thin rod made of stainless steel with a flat disc at one end, having a diameter larger than the diameter of the rod portion. The disc shape 10 at the end of the pusher 12 is advantageous as it helps to disperse the pressure on the practitioner's hand as the rod 12 is pushed through the needle 16.

In the embodiment shown in FIG. 4A, the needle 16 has a feeding/handling end 17, where a suture 7 and the pusher 12 are introduced into the interior channel 5 through a first aperture 19. Similarly to the embodiment shown in FIG. 1, the needle has a pointed end 3 for inserting into the human or animal body. Proximal to but spaced away from the pointed end 3, is a second aperture (side hole) 18 out of which the suture 7 can be pushed.

As shown in FIG. 4A, the feeding end 11 has a receiving portion 17 arranged to receive a suture 7 into the first aperture 19 of the needle 16. The receiving portion 17 surrounds the needle body at the feeding end 11 and has a conical shape having a narrower end and a wider end. The narrower end is oriented in the direction of the pointed end 3 of the needle 16, with a wider base at the feeding end 11. The wider base has a lip that extends out into a larger disc shape. The receiving portion 17 helps to improve the ergonomics of the needle. Here, the receiving portion 17 helps to introduce the suture 7 and/or pusher 12 into the interior channel via the first aperture 19 at the feeding end 11, and helps a user to handle the needle 16.

FIG. 4B shows an alternate view of the pusher 12 and the needle 16, looking from the handling/feeding end 11 of the needle 16 towards the pointed end 3 of the needle 16, such that the tip of the rod portion of the pusher 12 and pointed end of the needle 16 are directed into the page. The view in FIG. 4B thus faces the disc shaped end 10 of the pushing rod and the handling/feeding end 11 of the needle 16. The distal end of the receiving portion 17 at the handling/feeding end 11 of the needle 16 takes the shape of a disc with a small hole or aperture 19 arranged to receive a suture 7 and the pushing rod 12.

FIG. 5A shows the needle 16 of FIG. 1 in use, in combination with the suture 7 of FIG. 3 and the pushing rod 12 of FIG. 4A.

The suture deployment apparatus 2 comprises the needle 16, a T-bar suture 7 and a pushing rod 12. The deployment takes place once the needle 16 has been inserted, such that the side-hole 19 is situated within the target tissue of a human or animal body, as will be described in more detail below.

In operation, first the T-bar suture 7 is placed in the interior channel 5 of the needle 16. The flexibility of the thread 8 allows the suture 7 to bend so that the longitudinal axis of the footplate 6 aligns with the longitudinal axis of the needle 16. The suture 7 is inserted into the first aperture 19 of the needle 16, followed by the pusher 12 which is then gradually fed through the interior channel 5 of the needle 16.

As can be seen in FIG. 5A, the interior channel 5 is sized and shaped to receive both the thread 8 of the suture 7 and the pushing rod 12, side by side within the interior channel 5. The cross-sectional area of the interior channel 5 is sized to be spacious enough such that the pushing rod 12 and thread 8 can align without getting stuck, but tight enough that the pushing rod 12 is always situated behind the footplate 6 of the suture 7, and does not slip over the footplate 6.

As the pusher 12 pushes the suture 7 through the interior channel 5, the footplate 6 approaches the curved surface 14 which directs the footplate 6 out of the second aperture (side-hole) 18.

The step of the process where the footplate 6 of the T-bar suture 7 exits from the side-hole 18 is shown in FIG. 5B.

The pushing rod 12 is pushed through the needle 16 such that the tip of the pushing rod 12 (distal from the handling/feeding end 10) reaches the side-hole 18. The T-bar suture 7 is shown exiting the side-hole 18 at an angle to the longitudinal axis of the needle 16. The curved surface 14 results in smooth delivery of the suture 7, reducing the likelihood of the suture 7 snagging or stopping when the suture 7 is exiting the aperture 18.

Having the second aperture 18 at the side of the needle 16, in combination the curved surface 14, causes the T-bar suture 7 to be deployed into the soft tissue substantially perpendicular to the longitudinal axis of the needle 16. This is advantageous as the footplate 6 can be anchored better into the target tissue when the footplate 6 is angled such that the longitudinal axis of the footplate 6 is approximately perpendicular to the direction the thread 8 will be pulled. When the suture 7 is secured under tension, and the thread 8 is pulled, it is less likely to move around in or slip through the target tissue than other types of suture.

In FIG. 5B it can be seen that, owing to the topology of the apparatus 2, the suture 7 is deployed at a specified distance 4 from the pointed end 3.

The apparatus 2 could be used for a variety of procedures that may involve depositing a suture 7 into the soft tissue of an animal or human body. However, in a preferred embodiment, the apparatus 2 is used for the treatment of stress urinary incontinence in females. The procedure for treating stress urinary incontinence will 35 be described with reference to FIGS. 6, 7, 8, 9A, 9B, 10A, and 10B.

The procedure described is similar to laparoscopic colposuspension, however, the apparatus 2 enables the treatment to be completed more efficiently, using only local anaesthetic. The procedure described may be more minimally invasive and less morbid in comparison with existing treatments for SUI. It is envisaged that the procedure described will make treatment easier to perform such that a larger number of practitioners (including radiologists and surgeons), not essentially trained in laparoscopic surgeries, are able to perform minimally invasive SUI treatments.

FIGS. 6-10B show cross-sectional views of a human female pelvis in the transverse plane. The patient undergoing the treatment is lying down on their back. The view shown in FIGS. 6-10B is similar to what would be viewed by a practitioner from a screen displaying a real-time CT image. This procedure may be combined with fluoroscopic and CT facilities to help the practitioner guide the needle 16 to the correct part of the body.

In FIGS. 6-8 the urethra 20 and vaginal wall 22 are depicted schematically. The target tissue, in this case, is the periurethral tissue of the vaginal wall 22. FIGS. 6-8 show various steps of the procedure according to an embodiment of the invention.

FIG. 6 shows that the needle 16 has been inserted at an approximately 45° angle to the floor, directed at one side of the vaginal wall 22. In this example, the needle 16 is advanced under fluoroscopic imaging through the obturator foramen; however, other approaches may be used (e.g. inferior to the pubic bone or through the pubis symphysis).

Before the needle is inserted, a 6-8 French (1 French=⅓ mm) urinary catheter may be used to fill the bladder with a dilute contrast agent (then a concentrated contrast agent (3 ml)) to show the catheter tubing better under fluoroscopy. The practitioner may then manipulate the image intensifier with appropriate craniocaudal, right anterior oblique (RAO) and/or left anterior oblique (LAO) tilt, until a clear path can be seen (not overlaid by bony or vascular structures) and the catheter in the mid-urethra can be seen on a viewing screen. The practitioner then centres the display and may also use laser crosshairs to mark the incision/puncture area. The local anaesthetic would then be infiltrated in-line with the laser crosshairs with a spinal needle down to the vaginal wall 22 to the ipsilateral side of the urethra (not pictured). Ultrasound imaging may also be used during the procedure to ensure that important soft tissue structures such as blood vessels and nerves are not in line with the path of the needle.

To insert the needle 16 into the position shown in FIG. 6, a small skin incision is made at the desired puncture site. The skin incision is to be used for burying sutures at the end of the procedure. The flat disc surface at the feeding end of the needle 16 may be useful for allowing laser crosshairs to be seen clearly, which may help with the alignment of the central interior channel 19 perpendicular to the surface of the image intensifier (i.e. along the line of sight, so that the needle appears as just a dot on the image). The needle 16 is fed through—under fluoroscopy—until the pointed end 3 reaches the anterior wall of the vagina 22 adjacent to the urethra 20 and just below the bladder.

The needle 16 may pass through the obturator foramen to reach this position or via other approaches mentioned above. Digital vaginal examination may be performed synchronously to ensure that the vaginal wall is not traversed. The needle 16 should pass close to the periosteum where there is strong ligamentous tissue for suture 7 fixation. The target tissue (where the sutures are to be placed), in this procedure, is the periurethral anterior vaginal wall tissue.

FIG. 7 shows a variation to the procedure shown in FIG. 6, in which a solid object 24, in the form of a smooth plastic cylinder 24, which has been inserted into the patient's vagina. The pointed end 3 of the needle 16 is inserted into the tissue until it comes into contact with the solid object. Then, while the needle 16 is being pressed against the solid object 24, the T-bar suture 7 is deployed. This ensures that the suture 7 is placed accurately at the desired distance defined by the spacing 4 between the pointed end 3 and the second aperture 18. The presence of the solid object 24, also prevents the needle 16 traversing the vaginal wall 22.

The suture 7 is pushed down through the needle 16, with a pushing rod 12 pushing the suture 7 through the interior channel 5 of the needle 16 until the footplate 6 of the suture 7 exits the second aperture 18 and is retained in the periurethral anterior vaginal wall tissue, as illustrated in FIGS. 5A and 5B. One or more sutures 7 may be placed at a time on each side. The angle of deployment may be altered between sutures by rotating the body of the needle 16 (and hence the second aperture 18).

The needle 16 and pusher 12 are then removed, leaving the suture 7 in position in the tissue, and the process is repeated on the other side, with another suture 7 deployed in the vaginal wall 22, entering through the groin again but on the other side of the body. FIG. 8 shows the sutures 7 in place after the needle 16 and pusher 12 are removed. Only two T-bar sutures 7 have been placed into the vaginal wall 22 in this example, however, this process could be repeated a number of times.

Turning back to FIG. 4A, the feeding end of the needle 16 has arrow markings which indicate the side of the needle 16 having the second aperture 18, which is the side that the suture 7 will exit from. This may be helpful, for example, if the procedure involves depositing one suture 7 toward the head, and then rotating the needle 180° in situ before depositing another suture 7 toward the feet. This head-to-toe positioning would be preferable as it would lessen the likelihood of damaging the urethra 20. The arrow markings 17 allow the practitioner to know in which direction the suture 7 will be deployed, which is especially helpful when deploying two or more sutures 7 in succession.

FIGS. 9A-10B show two methods of securing the free end 8 of the suture 7 under tension, once it has been deployed as shown in FIGS. 6-8. This step of the process provides the urethra 20 with support, similarly to colposuspension, which may ameliorate the symptoms of SUI.

In FIG. 9A, a securing device (crimp) 26 is used to secure the threads 8 of both of the sutures 7. Here, in the trans-obturator approach, the sutures 7 are secured under tension in the same area in front of the pubis symphysis and held together by a shared crimp 26. The suture threads 8 are brought to the midline position using a tunnelling tool. The crimp 26 could be any material, but in this example the crimp is made of stainless steel.

An alternative approach of the needle would be inferior to the pubic bone (not pictured). This approach may require a shorter needle inserted at a narrower angle and helps to reduce the risk of injury to the femoral vessels/nerves.

In FIG. 9A, a common crimp 26 is used to secure the threads 8 of both of the sutures 7. The crimp 26 could be any material, but in this embodiment the crimp is made of stainless steel. The suture threads 8 are directed to the midline position using a tunnelling tool, and are brought out of the body through the same incision. FIG. 9B shows the crimp 26 in its final position. At this point the T-bar sutures 7 are secured around the pubic bone under tension, thus keeping the vaginal wall in place and providing support to the urethra 20.

FIGS. 10A and 10B show a variation of the superficial securing method shown in FIGS. 9A and 9B.

In FIG. 10A a first crimp 28 and a second crimp 28 are used to respectively secure each of the threads 8 of the sutures 7. Two crimps are needed for this method but they are likely to be the same type of crimp and may be identical to the crimp from FIGS. 9A and 9B, or made of a plug of absorbable material (e.g. collagen) that can only advance along the thread towards the T-bar and is restrained from slipping back in the opposite direction. The suture threads 8 are held in position by each crimp 28.

The same incision that was made for the needle is used for the crimps. In this case, tunnelling to the midline is not necessary. FIG. 10B shows the final position of the sutures according to the second method of superficially securing the sutures 7 under tension. Similarly to FIG. 9B, the sutures 7 are secured under tension, thus keeping the vaginal wall 22 in place and providing support to the urethra 20.

An absorbable material may be used for the footplate 6 of the suture. The formation of natural scar tissue related to suture absorption would then cause the urethra to be supported, having lasting positive effects after the footplate 6 is absorbed. To achieve this, the footplate 6 could be made from a bio-absorbable polymer—e.g. a 50:50 blend of lactide and glycolide polymers. Additionally, the crimp 26, 28 may be made from a bio-absorbable material. The treatment may involve a completely absorbable system, such that the SUI treatment does not leave permanent synthetic material behind in the body. If the suture 7 is entirely absorbable, scar tissue would form along the whole suture tract and effectively replace the whole system (T-bar, suture, crimp).

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. A needle for deploying a suture, the needle comprising:
a feeding end comprising a first aperture for receiving the suture;
a pointed end for creating an incision, wherein the needle extends between the feeding end and the pointed end;
a second aperture proximal to and spaced from the pointed end; and
an interior channel extending between the first aperture and the second aperture for transporting the suture from the first aperture to the second aperture.

2. The needle as claimed in claim 1, wherein the pointed end comprises a bevel.

3. The needle as claimed in claim 1, wherein the second aperture extends through a wall of the needle.

4. (canceled)

5. The needle as claimed in claim 1, wherein the needle comprises markings indicating the side of the needle in which the second aperture is formed.

6. The needle as claimed in claim 1, wherein the particular distance is based on the thickness of a target human or animal tissue and/or the angle at which the end of the suture exits from the second aperture into the target tissue.

7. The needle as claimed in claim 1, wherein a portion of the needle between the pointed end and the second aperture is closed.

8. The needle as claimed in claim 1, wherein a portion of the needle between the pointed end and the second aperture is solid.

9. The needle as claimed in claim 1, wherein the interior channel comprises a deflection surface arranged to deflect a suture toward the second aperture when the suture is incident on the deflection surface.

10. The needle as claimed in claim 9, wherein the deflection surface is on a side of the second aperture proximal to the pointed end of the needle.

11. The needle as claimed in claim 9, wherein the deflection surface comprises one side of the wall of the interior channel, which optionally forms a curve toward an edge of the second aperture proximal to the pointed end of the needle.

12. The needle as claimed in claim 1, wherein the needle comprises a material that is visible through fluoroscopic imaging and/or CT imaging.

13. (canceled)

14. The needle as claimed in claim 1, wherein the needle is arranged to receive a pusher for pushing the suture through the interior channel of the needle and out of the second aperture.

15. A kit for deploying sutures, comprising:
at least one suture;
at least one needle according to claim 1; and
at least one pusher, arranged to push the suture through the interior channel of the needle and out of the second aperture.

16. The kit as claimed in claim 15, wherein the at least one suture is a T-bar suture.

17. The kit as claimed in claim 16, wherein each suture comprises a footplate portion and a thread portion.

18. (canceled)

19. The kit as claimed in claim 15, comprising a member for inserting into an orifice of the human or animal body, wherein the member is arranged to support the pointed end of the needle in a target human or animal tissue and wherein the member is cylindrical with a tapered end.

20. (canceled)

21. The kit as claimed in claim 15, wherein the suture comprises a free end and the kit comprises an anchoring device for securing the free end of each suture under tension at an anchoring point.

22. The kit as claimed in claim 15, further comprising a tunnelling device for pulling the thread portion or free end of each suture through tissue to an anchoring point.

23-25. (canceled)

26. A method for the treatment of stress urinary incontinence, the method comprising:
inserting the needle of claim 1, into a female human or animal body;
advancing the needle until the pointed end reaches a periurethral anterior vaginal wall tissue;
transporting the suture through the interior channel of the needle until the end of the suture exits the needle through the second aperture and the suture is retained in the periurethral anterior vaginal wall tissue;
removing the needle from the female human or animal body; and
securing the suture, under tension such that a pubourethral ligament is reinforced.

27. A method for the treatment of stress urinary incontinence, the method comprising:
transporting a T-bar suture into a periurethral anterior vaginal wall tissue of a female human or animal body such that the T-bar suture is retained in the periurethral anterior vaginal wall tissue; and
securing the T-bar suture, under tension such that a pubourethral ligament is reinforced.
